(19) United States
(12) Patent Application Publication
(10) Pub. No.: US 2014/0148399 A1
(43) Pub. Date: May 29, 2014

(54) Compositions and methods for treating or ameliorating obesity or for reducing diabetic hypercholesterolemia

(71) Applicants: UNIVERSITY OF TSUKUBA, Tsukuba-Shi (JP); CENTER OF BIOTECHNOLOGY OF BORJ CEDRIA, Hammam Lif (TN)

(72) Inventors: Hiroko Isoda, Tsukuba-Shi (JP); Junkyu Han, Tsukuba-Shi (JP); Zar Kalai Feten, Tsukuba-Shi (JP); Chedly Abdelly, Hammam Lif (TN); Riadh Ksouri, Hammam Lif (TN)

(73) Assignees: CENTER OF BIOTECHNOLOGY OF BORJ CEDRIA, Hammam Lif 2050 (TN); UNIVERSITY OF TSUKUBA, Tsukuba (JP)

(21) Appl. No.: 13/685,104

(22) Filed: Nov. 26, 2012

(51) Int. Cl. A61K 36/185; A61K 31/7048

(52) U.S. Cl. CPC A61K 36/185; A61K 31/7048
USPC 514/27; 435/377; 435/375

(57) ABSTRACT

Obesity is one of the major health concerns in the Twenty-First Century and is one of the leading causes of preventable death. It is a strong risk factor for Type 2 Diabetes. Disclosed herein are compositions and methods using Nitraria retusa extracts for enhancing energy metabolism, inhibiting fat accumulation, inhibiting preadipocyte differentiation, reducing diabetic hypercholesterolemia, and for treating or ameliorating obesity.

### BACKGROUND OF THE INVENTION

For many years, a diet rich in vegetables and fruits has been recognized as protective against chronic diseases such as obesity, diabetes, atherosclerosis, hypertension and cancer. This diet has been implicated in mediating glucose transport in in vitro and in vivo studies. Scientific research is constantly looking for new molecules that could be used as dietary functional ingredients in the fight against obesity and diabetes, two pathologies highly prevalent in Western societies. Flavonoids are a group of molecules of increasing interest in this regard. Recent investigations of adipose tissue biology have led to an improved understanding of the mechanisms linking obesity with metabolic syndrome and other complications.

Adipocytes play a central role in the maintenance of lipid homeostasis and energy balance in vertebrates by storing triglycerides or by releasing free fatty acids in response to changes in energy demands. However, obesity is associated with a number of pathological disorders such as Type 2 Diabetes Mellitus, hypertension, hyperlipidemia, and cardiovascular diseases. Obesity, defined as an excess of adipose tissue when body mass index (BMI) is ≧30 kg/m², is due to an imbalance between energy intake and energy expenditure. Obesity is not only caused by adipose tissue hypertrophy, but also by adipose tissue hyperplasia, which triggers the transformation of pre-adipocytes into adipocytes. However, the molecular basis for these associations remains to be elucidated, which causes the search for anti-obesity agents to be difficult.

The loss of insulin action selectively in adipose tissue leads to secondary disorders of diabetes through the malfunctioning of adipocyte function, changes in adipogensis alterations in glucose and lipid metabolism, and protein expression. The strategy to reduce hyperglycemia without increasing adiposity or with reduction of body weight constitutes a preferred mechanism for a drug for treating Type 2 Diabetes.

Type 2 Diabetes is closely associated with other metabolic disorders, such as hypertension, cardiovascular diseases, and atherosclerosis and its incidence is increasing worldwide. Insulin resistance is an important marker for developing Type 2 Diabetes. The roles of life-style changes and weight loss in preventing diabetes have been proven in clinical trials. However, more effective and safe medicines are needed.

Recently, the clinical importance of herbal drugs has received considerable attention. One example are the flavonoids, which are often found in herbal drugs and foods; they are known to possess many biological properties. Previous studies have demonstrated that flavonoids and/or flavonoid-rich fractions ameliorate experimental diabetes in mice and rats. Several flavonoids were reported to inhibit insulin signaling in adipocytes.

The benefits of a pharmaceutical to aggressively treat a disease in its early stages is preferable, but medications may have unwanted side effects. In this context, flavonoids, among which quercetin is one of the most commonly found in foods, have been reported to improve diabetic status.

Adipose tissue is not only a storage organ for triglycerides but also is an endocrine organ where numerous chemical messengers called adipokines are released for communicating with other tissues. Adipokines include adiponectin which promotes cell proliferation as well as the differentiation of preadipocytes into adipocytes. Thus, adipokine release augments the programmed gene expression that is responsible for adipogenesis and increasing lipid content in adipocytes.

Thiazolidinedione-type compounds, such as pioglitazone, are major ligands of peroxisome proliferator-activated receptor gamma (PPARγ) and potent insulin sensitizers; they are known to and increase the levels of adiponectin, an important adipokine associated with insulin sensitivity in adipose tissue. These compounds are currently used clinically to treat Type 2 Diabetes. It is well established that PPARγ agonists such as thiazolidinedione-type compounds promote the adipogenesis of 3T3-L1 cells. Thus, these cells have been used for screening potential anti-diabetic compounds. The murine 3T3-L1 cell line, which is widely used as a cell model, has been a mainstay for adipose cell biology research over several decades.

Reactive oxygen species (ROS) play in important role in oxidative stress related to the pathogenesis of various important diseases. In healthy individuals, the production of free radicals is balanced by the antioxidative defense system. Knowledge and application of such potential antioxidant activities in reducing oxidative stresses in vitro has revealed cost-effective antioxidants from various plant sources. Phytochemicals are secondary metabolic products produced by plants in response to the environmental stresses. Laboratory studies have demonstrated that some plants when eaten in whole or their active constituents are taken in isolation, provide adequate protective effects against human carcinogenesis and mutagenesis. Also, herbal remedies and phytotherapy drugs are currently developed to protect against electrophile (e.g., free radical) attack on DNA; such electrophilic attack on DNA has widespread outcomes, such as ageing and cancer.

The genus Nitraria is a shrub that bears edible berries and is well adapted to arid climates. Species of Nitraria are found in desert regions of South-East Europe (N. komaroui and N. sibirica), in the Middle-East (N. schoberi), in the desert areas of Qinghai-Tibetan Plateau (N. tangutorum), in Australia (N. billardieri), and in Africa, particularly in northern and occidental parts of the Sahara desert and in Mauritania (N. tridendata and N. retusa).

Nitraria fruits have been used as nutritional food and traditional herb for the treatment of hypertension, abnormal menstruation, and indigestion among folks in the northwest of China. There, N. tangutrun fruits are added to the daily diet of lactating women to help milk production.

Nitraria retusa Forssk is known in Tunisia as “Ghardaq” and is used by the Bedouins as a source of fuel. Its fleshy red fruits are eaten by humans and are used to prepare drinks. The leaves serve as a supplement for tea and are used as poultice. The ashes of this species have the ability to remove fluids of infected wounds. Fresh leaves of Nitraria retusa decoction is used in Morocco in case of poisoning, upset stomach, ulcers, gastritis, enteritis, heartburn, colitis, and colonic abdominal pain. Nonetheless, it is unknown whether Nitraria retusa extracts are effective in inhibiting preadipocyte differentiation, reducing diabetic hypercholesterolemia, inhibiting lipid droplet accumulation in an adipocyte, or enhancement of energy metabolism in the liver and are generally effective in treating or ameliorating obesity.

### SUMMARY OF THE INVENTION

As described below, the present invention features compositions and methods for inhibiting preadipocyte differentiation, reducing diabetic hypercholesterolemia, inhibiting lipid droplet accumulation in an adipocyte, and enhancement of energy metabolism in the liver and generally for treating or ameliorating obesity.

In one aspect, the invention provides a composition formulated for oral administration including a therapeutically effective amount of a Nitraria retusa extract or a purified fraction thereof.

In another aspect, the invention provides a method for inhibiting preadipocyte differentiation, the method includes administering to a preadipocyte an effective amount of a Nitraria retusa extract or a purified fraction thereof. In one embodiment, the preadipocyte is in vitro. In another embodiment, the preadipocyte is in a subject being a human, a pet, or a livestock. In yet another embodiment, inhibiting fat accumulation in the preadipocyte treats or ameliorates obesity, and/or it increases fat burning.

In yet another aspect, the invention provides a method for reducing diabetic hypercholesterolemia, the method including administering to a subject in need thereof an effective amount of a composition comprising Nitraria retusa extract or a purified fraction thereof.

In still another aspect, the invention provides a method for inhibiting lipid droplet accumulation in an adipocyte, the method includes administering to an adipocyte an effective amount of a Nitraria retusa extract or a purified fraction thereof.

In another aspect, the invention provides a method for enhancement of energy metabolism in the liver, the method including administering to a subject in need thereof an effective amount of a composition comprising Nitraria retusa extract or a purified fraction thereof. In an embodiment, enhancement of energy metabolism in the liver is enhancement of gene expression related to lipid metabolism, e.g., Fatty Acid Synthase (FAS), peroxisome proliferator-activated receptor alpha (PPAR-α), peroxisome proliferator-activated receptor gamma (PPAR-γ), or sterol regulatory element binding protein-1c (SREBP-1c). In another embodiment, enhancement of energy metabolism in the liver treats or ameliorates obesity.

In any of the above aspects or embodiments, the extract is an ethanol extract. In any of the above aspects or embodiments, the composition includes a therapeutically effective amount of a Nitraria retusa extract. In any of the above aspects or embodiments, the composition includes a therapeutically effective amount of a purified fraction of a Nitraria retusa extract. In any of the above aspects or embodiments, the composition includes an isolated compound. In any of the above aspects or embodiments, the Nitraria retusa extract or a purified fraction thereof is in a composition being a food product, a dietary supplement, or a pharmaceutical composition and being formulated for oral administration. The food product is one or more of the following: beverage, bread, candy, cereal, chocolate, coffee, condiment, cookie, cracker, energy drink, gel, ice cream, jelly, juice, milk-containing beverage, nutritional bar, pasta, paste, processed fruit, processed grain, processed meat, processed vegetable, pudding, snack bar, soft drink, tea, yogurt, animal feed, or pet food. In some embodiments, the food product is a tea.

In any of the above aspects or embodiments, the composition is formulated as a tablet, a capsule, a softgel, a liquid gel, a pill, a granule, a syrup, a paste, a powder, a lozenge, a concentrate, a liquid, or a dry syrup.

In any of the above aspects or embodiments, the subject is a human, a pet, or a livestock. In some embodiments, the subject is a human who is a diabetic or a prediabetic.

The above aspects and embodiments can be combined in an possible manner.

Other features and advantages of the invention will be apparent from the detailed description and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Nitraria retusa extract or purified fraction thereof” is meant one or more compounds which are normally present in a sample of Nitraria retusa and which can be extracted, isolated, or solubilized from the sample of Nitraria retusa. A Nitraria retusa ethanol extract can be a first product obtained by combining a dried Nitraria retusa sample with ethanol; thus, the one or more compounds can remain in an ethanol solution. Alternately, the first product obtained by combining a dried Nitraria retusa sample with ethanol can be evaporated to produce a dried product; thus, the Nitraria retusa ethanol extract contains the one or more compounds but substantially no ethanol. Alternately, ethanol and/or the one or more compounds can be removed from the first product using other methods known in the art. Moreover, a Nitraria retusa extract can be further purified into a plurality of distinct fractions thereof, each of which may have a therapeutic benefit. The fractions can be further purified into distinct isolated compounds.

By “adipogenesis” is meant an increase in the number of adipocytes. Adipogenesis typically involves hyperplasia (increase in number) of adipocytes. Adipocyte hypertrophy is the increase in size of a pre-existing adipocyte as a result of excess triglyceride accumulation, i.e., lipid droplet or fat accumulation. Hypertrophy occurs when energy intake exceeds energy expenditure or energy metabolism, e.g., fat burning. Hyperplasia results from the formation of new adipocytes from precursor cells (preadipocytes) in adipose tissue. Typically hyperplasia involves the proliferation of preadipocytes and their differentiation into adipocytes.

By “metabolic syndrome” is meant one or more risk factors that increase a subject's propensity to develop coronary heart disease, stroke, peripheral vascular disease and/or type II diabetes. Risk factors associated with metabolic syndrome include abdominal obesity (i.e., excessive fat tissue in and around the abdomen, atherogenic dyslipidemia including but not limited to high triglycerides, low high-density lipoprotein (HDL) cholesterol and high low-density lipoprotein (LDL) cholesterol, elevated blood pressure, insulin resistance or glucose intolerance, prothrombotic state (e.g., high fibrinogen or plasminogen activator inhibitor-1 in the blood), proinflammatory state (e.g., elevated C-reactive protein in the blood). Compositions and methods of the invention are useful for the treatment or prevention of metabolic syndrome.

By “obesity” is meant an excess of body fat relative to lean body mass. A human subject is considered obese if they have a body mass index (BMI) of 30 and above. However, the term “obesity” is not limiting and subjectively depends on cultural and social definitions. Compositions and methods of the invention are useful for the treatment or prevention of obesity.

By “body mass index (BMI)” is a subject's weight in kilograms divided by their height in meters squared.

By “diabetic” is meant a subject having high blood sugar, either because the pancreas does not produce enough insulin, or because its cells do not respond to the insulin that is produced. This high blood sugar produces the classical symptoms of polyuria (frequent urination), polydipsia (increased thirst) and polyphagia (increased hunger). In humans, there are three main types of diabetes mellitus. Type 1 Diabetes Mellitus results from the body's failure to produce insulin. Type 2 Diabetes Mellitus results from insulin resistance, a condition in which cells fail to use insulin properly, sometimes combined with an absolute insulin deficiency. This form was previously referred to as non insulin-dependent diabetes mellitus (NIDDM) or “adult-onset diabetes”. The third main form, gestational diabetes occurs when pregnant women without a previous diagnosis of diabetes develop a high blood glucose level. It may precede development of Type 2 Diabetes Mellitus. Other forms of diabetes include congenital diabetes, which is due to genetic defects of insulin secretion, cystic fibrosis-related diabetes, steroid diabetes induced by high doses of glucocorticoids, and several forms of monogenic diabetes. In humans, diabetes mellitus is characterized by recurrent or persistent hyperglycemia, and is diagnosed by demonstrating any one of the following: fasting plasma glucose level ≧7.0 mmol/l (126 mg/dl); plasma glucose ≧11.1 mmol/l (200 mg/dL) two hours after a 75 g oral glucose load as in a glucose tolerance test; symptoms of hyperglycemia and casual plasma glucose ≧11.1 mmol/l (200 mg/dl); and glycated hemoglobin (Hb A1C) ≧6.5%. Compositions and methods of the invention are useful for the treatment or prevention of diabetes.

By “prediabetic” is meant a subject at risk for progression to full-blown diabetes. Prediabetes is the state in which some but not all of the diagnostic criteria for diabetes are met. It is often described as the “gray area” between normal blood sugar and diabetic levels. In humans, the subject not yet demonstrated an above-noted diagnosis criteria for diabetes mellitus. For example, a prediabetic can have plasma glucose at or above 140 mg/dL (7.8 mmol/L), but not over 200 mg/dL (11.1 mmol/L), two hours after a 75 g oral glucose load. A prediabetic is characterized by impaired glucose tolerance. Compositions and methods of the invention are useful for preventing, slowing, or lessening the progression of prediabetes into diabetes

By “hypercholesterolemia” is meant increased levels of total serum cholesterol or increased levels of low-density lipoprotein (LDL) cholesterol (the “bad cholesterol”).

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of a compound, molecule, nucleic acid, or protein to be detected.

By “disease” is meant any condition or disorder that damages or interferes with the normal function of a cell, tissue, or organ.

By “effective amount” is meant the amount of a required to ameliorate the symptoms of a disease relative to an untreated patient. The effective amount of active ingredient(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “extracted”, “isolated,” “purified,” or “biologically pure” refer to material that is free to varying degrees from components which normally accompany it as found in its native state. “Extract” and “isolate” denote a degree of separation from original source or surroundings. “Purify” denotes a degree of separation that is higher than isolation. A “purified” or “biologically pure” compound is sufficiently free of other materials such that any impurities do not materially affect the biological properties of the compound or cause other adverse consequences. That is, a compound is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography.

By an “isolated compound” is meant a compound that has been separated from components that naturally accompany it. Typically, the compound is isolated when it is at least 60%, by weight, free from the naturally-occurring organic molecules with which it is naturally associated. Preferably, the preparation is at least 75%, more preferably at least 90%, and most preferably at least 99%, by weight, of a desired compound. An isolated compound of the invention may be obtained, for example, by extraction from a natural source, by expression of a recombinant nucleic acid encoding such a compound, or by chemically synthesizing the compound.

As used herein, “obtaining” includes collecting, harvesting, purchasing, synthesizing, or otherwise acquiring matter.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, feline, or rodent.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Ranges from 50 to 100 include similar steps. Ranges between 100 and 200, 200 and 300, 300 and 400, and so forth also include similar steps.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any composition or method provided herein can be combined with one or more of any of the other compositions and methods provided herein.

### BRIEF DESCRIPTION OF THE DRAWINGS

FIGS. 1A and 1B are graphs showing the effects of (A.) Nitraria retusa ethanol extract and (B.) Nitraria retusa water extract on lipid droplet accumulation in 3T3-L1 cells.

FIG. 2 includes micrographs showing the effects of Nitraria retusa ethanol extract (“NR EtOH”) and water extract (“NR w”) of varying doses (25, 50, 100, 200, or 400 μg/ml) on 3T3-L1 preadipocyte cell differentiation and cell morphology after 9 days. Isorhamnetin is a positive control. Circles (in treatment groups) point out hypertrophic cells having decreased lipid droplet size.

FIG. 3 is a graph showing the effects of Nitraria retusa extract administration (50 mg/kg per dose) on body weight of mice.

FIGS. 4A and 4B are graphs showing the effects of Nitraria retusa extract administration (50 mg/kg dose) on (A.) white adipose tissue weight and (B.) liver weight of mice.

FIGS. 5A-5E are graphs showing biochemical effects in mice administered 4 weeks of a Nitraria retusa extract: (A.) Total cholesterol levels, (B.) LDL-cholesterol levels, (C.) HDL-cholesterol levels, (D.) Glucose levels, and (E.) Triglyceride levels.

FIGS. 6A-6F are graphs showing gene expression effects in mice administered a Nitraria retusa extract: (A.) PPAR gamma levels, (B.) PPAR alpha levels, (C.) Acetyl-CoA carboxylase levels, (D.) Fatty acid synthase levels, (E.) SREBP-1c levels, and (F.) Lipoprotein lipase levels.

FIGS. 7A-7F are LC-MS Chromatograms. Experimental groups (A.-C.) show compounds present in Nitraria retus extracts. Control groups (D.-F.) confirm that the peaks in A.-C. represent Isorhamnetin-3-O-glucoside and Isorhamnetin 3-O-rutinoside.

### DETAILED DESCRIPTION OF THE INVENTION

The invention features compositions and methods that are useful for enhancing energy metabolism, inhibiting fat accumulation, inhibiting preadipocyte differentiation, reducing diabetic hypercholesterolemia, and for treating or ameliorating obesity.

The invention is based, at least in part, on the following in vitro and in vivo discoveries. First, shoot extracts of the edible halophyte Nitraria retusa were tested for their effects on biological activities such as lipid accumulation and preadipocyte cell differentiation using in vitro assays with a 3T3-L1 cell line. Here, differentiated 3T3-L1 cells were treated every two days with different extracts at various concentrations for seven days and more. Oil red O staining was performed to assay lipid accumulation in 3T3-L1 cells. Second, Nitraria retusa extracts were tested for their effects in in vivo assays using a diabetic mouse model. Here, effects on the body weight and organ weight (liver and adipose tissue) were measured for mice exposed and unexposed to Nitraria retusa extracts. Also, expression levels of genes critical to lipid or cholesterol metabolism were assayed.

These discoveries indicate that Nitraria retusa extracts enhance energy metabolism, inhibit fat accumulation, inhibit preadipocyte differentiation, reduce diabetic hypercholesterolemia, and treat or ameliorate obesity.

The therapeutic methods of the disclosure, which include prophylactic treatment, in general comprise administration of a therapeutically effective amount of a composition described herein, to a subject (e.g., animal and human) in need thereof, including a mammal, particularly a human to produce a desired effect, i.e., treating or ameliorating emotional-psychological stress. Such treatment will be suitably administered to subjects, particularly humans, suffering from, having, susceptible to, or at risk for an emotional-psychological stress or having symptoms thereof. Identifying a subject in need of such treatment or determination of those subjects “at risk” can be in the judgment of a subject or a health care professional or veterinarian and can be subjective (e.g., opinion) or objective (e.g., measurable by a test, diagnostic method, family history, and the like).

Administration of a composition can be accomplished via a single or divided doses.

As defined herein, a therapeutically effective amount of composition (i.e., an effective dosage) depends on the formulation selected. The formulations can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present.

Human dosage amounts can initially be determined by extrapolating from the amount of compound used in an animal model (e.g., mice), as a skilled artisan recognizes it is routine in the art to modify the dosage for humans compared to animal models.

In certain embodiments it is envisioned that an in vivo dosage may vary from between about 1 mg of a Nitraria retusa extract/kg body weight to about 1000 mg of a Nitraria retusa extract/kg body weight, e.g., between about 5 mg/kg body weight to about 500 mg/kg body weight; from about 10 mg/kg body weight to about 400 mg/kg body weight; from about 25 mg/kg body weight to about 200 mg/kg body weight; or from about 50 mg/kg body weight to about 100 mg/kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000 mg/kg body weight.

The weight (in mg) of a Nitraria retusa extract represents the weight of dried Nitraria retusa shoots originally used to prepare a first product obtained by combining a dried Nitraria retusa sample with ethanol or hot water. Alternately, the weight (in mg) of a Nitraria retusa extract represents the weight of a dried product that is produced by evaporating (or otherwise excluding ethanol or water from) the first product by combining a dried Nitraria retusa sample with ethanol or hot water. Additionally, the weight (in mg) of a Nitraria retusa extract represents the weight of a dried fractionated product which is produced when a Nitraria retusa extract is further purified into a plurality of distinct fractions thereof. Finally, the weight (in mg) of a Nitraria retusa extract represents the weight of an isolated compounds isolated from a Nitraria retusa extract or fraction thereof.

In other embodiments it is envisioned that an in vitro dosage of a Nitraria retusa extract may vary from between about 1 μg of a Nitraria retusa extract per ml of culture medium to about 1000 μg of a Nitraria retusa extract per ml of culture medium, e.g., between about 5 μg/ml to about 500 μg/ml; from about 10 μg/ml to about 400 μg/ml; from about 25 μg/ml to about 200 μg/ml; or from about 50 μg/ml to about 100 μg/ml. In other embodiments this concentration may be about 1, 5, 10, 25, 50, 75, 100, 150, 200, 250, 300, 350, 400, 450, 500, 550, 600, 650, 700, 750, 800, 850, 900, 950, 1000 μg/ml. Again, the weight of a Nitraria retusa extract is as described above in the preceding paragraph.

Of course, the above-mentioned dosage or concentration amounts may be adjusted upward or downward, as is routinely done in such treatment protocols, depending on the results of the initial clinical trials and the needs of a particular patient.

Toxicity and therapeutic efficacy of such formulations can be determined by standard procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Formulations which exhibit high therapeutic indices can be preferred. While formulations that exhibit toxic side effects may be used, care should be taken to design a delivery system that targets such formulations to the site of affected tissue in order to minimize potential damage to uninfected cells and, thereby, reduce side effects.

The data obtained from the cell culture assays and animal studies can be used in formulating a range of dosage for use in humans. The dosage of such formulations optionally lies within a range of circulating concentrations that include the ED₅₀ with little or no toxicity. The dosage may vary within this range depending upon the dosage form employed and the route of administration utilized. For any formulation used in a method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations suitable for oral administration can consist of, e.g., (a) liquid solutions, such as an effective amount of the packaged cargo (e.g., nucleic acid) suspended in diluents such as water, saline, or PEG 400; (b) capsules, sachets, or tablets, each containing a predetermined amount of the cargo, as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and compatible carriers. Lozenge forms can comprise the cargo in a flavor, e.g., sucrose, as well as pastilles comprising the cargo in an inert base, such as gelatin and glycerin or sucrose and acacia emulsions, gels, and the like containing, in addition to the cargo, carriers known in the art.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; and (v) formulations that allow for convenient dosing, such that doses are administered, for example, once every one or two weeks. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The practice of the present invention employs, unless otherwise indicated, conventional techniques of biochemistry, botany, cell biology, food science, immunology, microbiology, molecular biology (including recombinant techniques), and nutrition which are well within the purview of the skilled artisan. Such techniques are explained fully in the literature.

### Examples

The present invention is described by reference to the following Examples. The following examples provide those of ordinary skill in the art with a complete disclosure and description of how to make and use the therapeutic methods of the invention, and are not intended to limit the scope of what the inventors regard as their invention. Standard techniques well known in the art or the techniques specifically described below were utilized.

### Example 1

### Exposure to Nitraria retusa Extracts Inhibits Lipid Droplet Accumulation In Vitro in a Dose-Dependent Manner

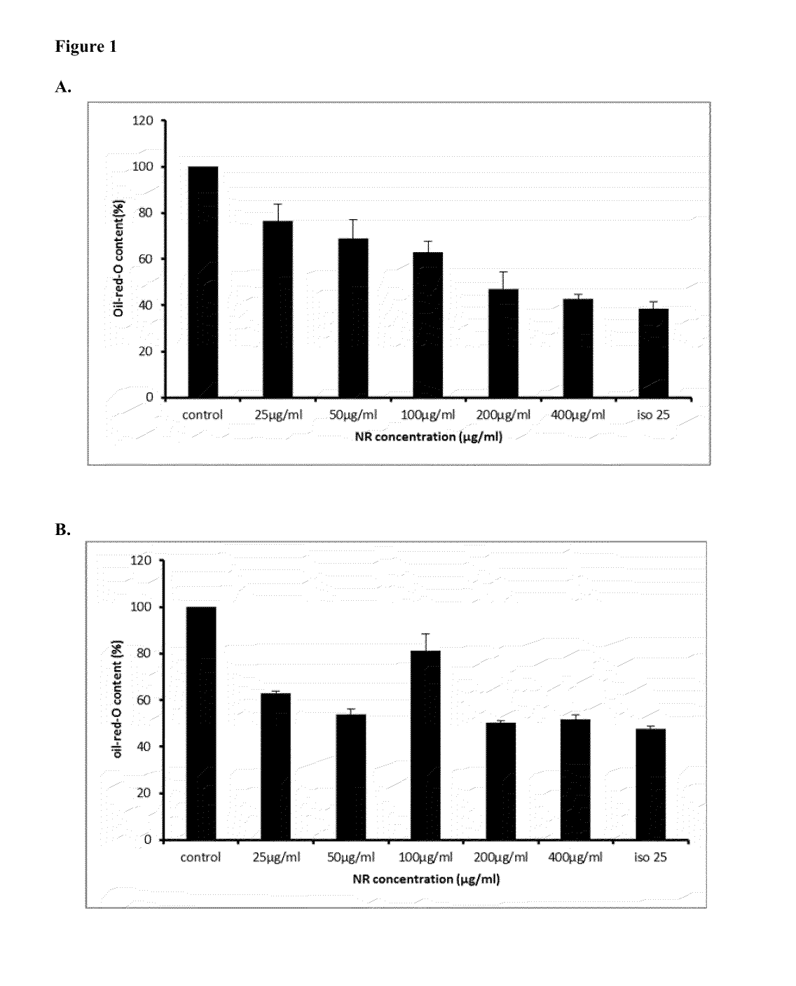

An adipogenesis assay was performed to investigate the effects of different concentrations of Nitraria retusa extracts on adipocyte differentiation and on the lipid droplets accumulation in vitro. For this, differentiated 3T3-L1 cells were treated every two days with extracts at various concentrations (25, 50, 100, 200, or 400 μg/ml) for seven days and more. Oil red O staining was performed to determine the lipid accumulation in 3T3-L1 cells. As shown in FIG. 1, both the ethanol Nitraria retusa extracts (A.) and the water Nitraria retusa extracts (B.) inhibited lipid droplet accumulation compare to control (detected by oil red O staining) in dose-dependent manners.

These data indicate that administration of a Nitraria retusa extract is effective in inhibiting fat accumulation in a preadipocyte, which treats or ameliorates obesity.

### Example 2

### Exposure to Nitraria retusa Extracts Inhibits Preadipocyte Differentiation but does not Inhibit Adipocyte Cell Proliferation In Vitro

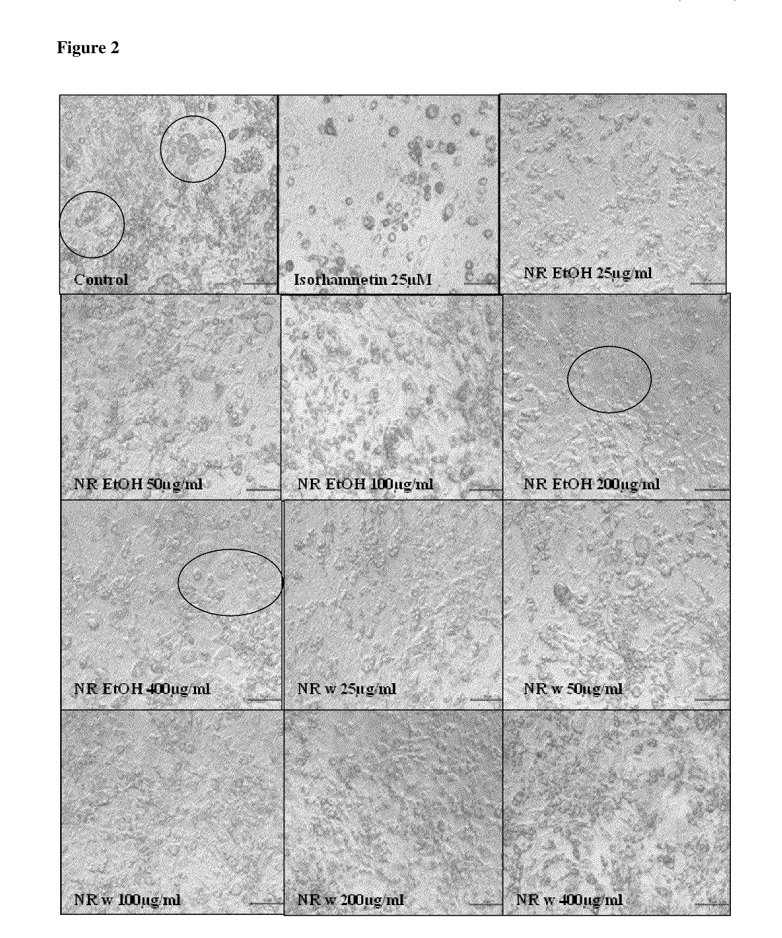

To confirm that Nitraria retusa extracts modulate adipocyte hypertrophy rather than hyperplasia (cell proliferation), in vitro adipocytes were exposed to Nitraria retusa extracts and were studied. Here, Isorhamnetin, a compound that inhibits adipogenesis by interfering with differentiation of adipose stem cells, served as a positive control. As shown in FIG. 2, Nitraria retusa extracts do not affect cell proliferation of adipocytes. Instead, Nitraria retusa extracts inhibit preadipocyte differentiation which is evident by modulation of the hypertrophy of cells (compare cells encircled in FIG. 2) and that the size of lipid droplet was decreased.

These data indicate that administration of a Nitraria retusa extract is effective in inhibiting preadipocyte differentiation, which treats or ameliorates obesity.

### Example 3

### Exposure to Nitraria retusa Extracts Reduces Adipose Tissue Weight in Diabetic Mice

To assay the in vivo effects of Nitraria retusa extracts, Nitraria retusa extract were administrated orally with a dose of 50 mg/kg body weight for thirty consecutive days. The dose was selected based on the above-mentioned toxicity study which used a ranged dose between 25 and 400 mg/kg body weight and which confirmed that a dose of 50 mg/kg body weight was safe.

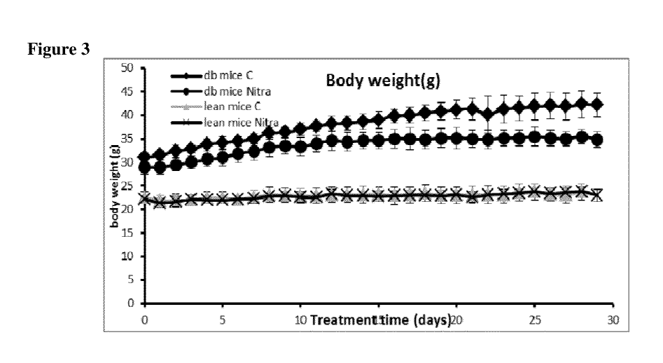

As shown FIG. 3 and Table 1 (below), oral administration of a Nitraria retusa extract significantly reduced body weight in diabetic mice models.

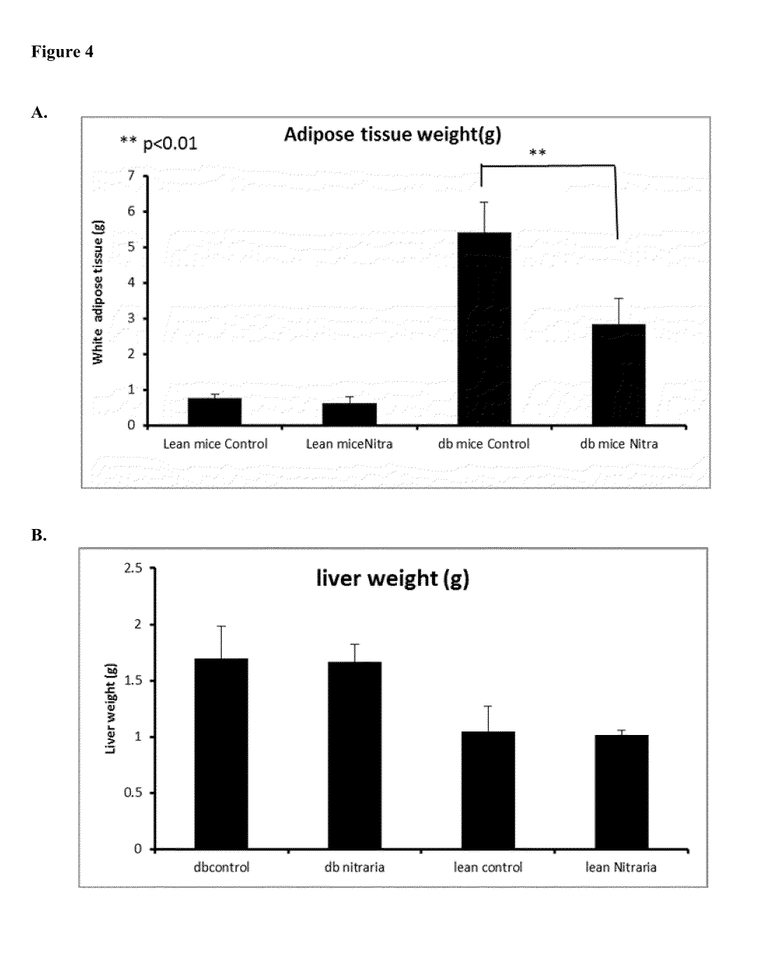

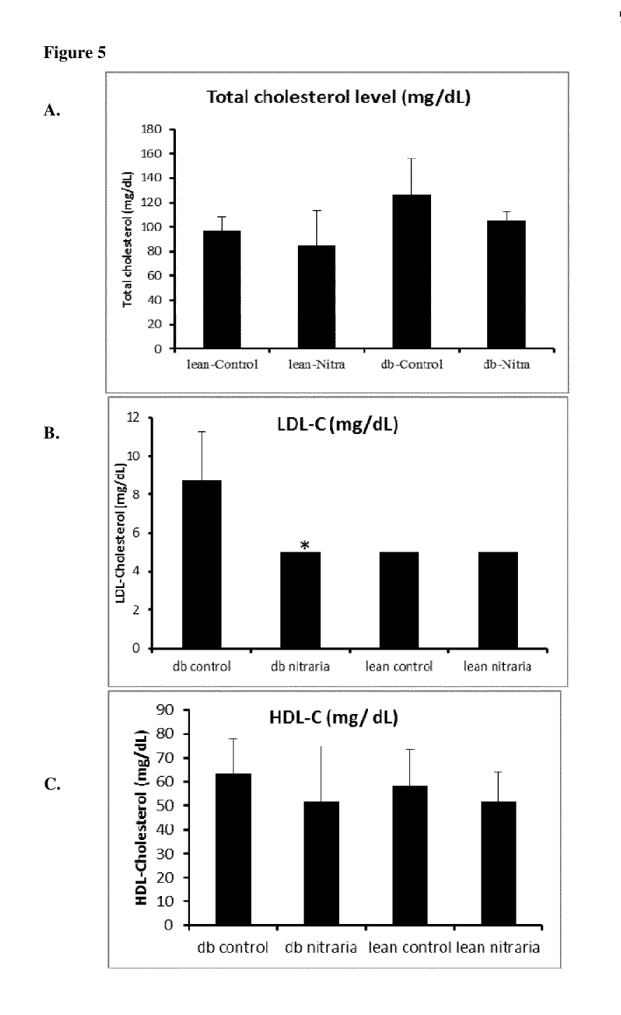

The weight loss is due to a significant decrease in overall adipose tissue weight in the Nitraria retusa extract exposed mice. As shown in FIG. 4A, the adipose tissue weight in diabetic control mice is significantly greater than the adipose tissue weight of diabetic mice administered Nitraria retusa extracts. However, as shown in FIG. 4B, administering Nitraria retusa extracts did not affect the liver's weight during the duration of the experiment.

TABLE 1
The effects of Nitraria retusa extract administration (50 mg/kg per dose)
on body weight and food intake of mice

Total Food intake

| | g/week | g/day |
|---|---|---|
| db mice-Control | 42.77 ± 7.59 | 6.1 ± 0.65 |
| db mice-Nitraria | 38.57 ± 5.1 | 5.51 ± 0.5 |
| Lean mice-Control | 25.17 ± 3.8 | 3.59 ± 0.69 |
| lean mice-Nitraria | 25.87 ± 4.75 | 3.69 ± 0.85 |

Body weight (g)

| | Initial | Final |
|---|---|---|
| db mice-Control | 31 ± 1 | 42.16 ± 2.56 |
| db mice-Nitraria | 29 ± 1.5 | 34.83 ± 1.75 |
| Lean mice-Control | 22.5 ± 1.22 | 23.6 ± 1.71 |
| lean mice-Nitraria | 22.2 ± 0.75 | 23 ± 1.32 |

These data indicate that in vivo administration of a Nitraria retusa extract is effective in treating or ameliorating obesity and/or increasing fat burning.

### Example 4

### Exposure to Nitraria retusa Significantly Reduces Low-Density Lipoprotein (LDL) Cholesterol (“Bad Cholesterol”) Levels in Diabetic Mice

An increase in triglyceride levels, particularly when accompanied by decreased high-density lipoprotein (HDL) levels, has been shown to be a surrogate marker for insulin resistance, a strong predisposing condition for Type 2 Diabetes.

Thus, the in vivo effects of Nitraria retusa extracts, on cholesterol levels (total, low-density lipoprotein (LDL) cholesterol, and HDL cholesterol) was assayed.

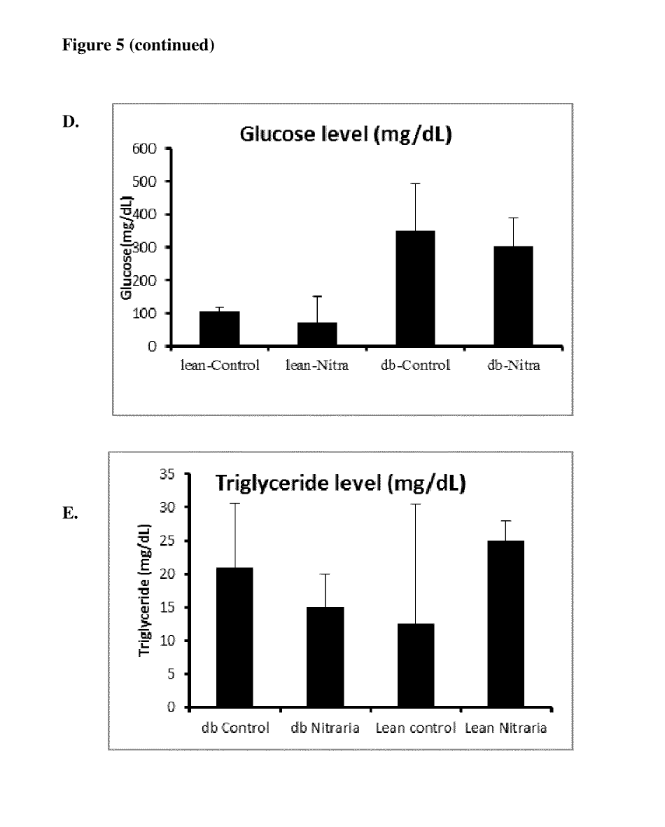

As shown FIGS. 5A and 5C, oral administration of a Nitraria retusa extract did not reduce levels of total cholesterol or levels of HDL cholesterol (the “good cholesterol”). However, as shown in FIG. 5B, oral administration of a Nitraria retusa extract significantly reduced LDL cholesterol (the “bad cholesterol”) levels in diabetic mice models.

As shown in FIGS. 5D and 5E, glucose and triglycerides levels were not significantly affected by administration of Nitraria retusa extracts. This is likely due to the duration of the disclosed experiments.

These data indicate that in vivo administration of a Nitraria retusa extract is effective in reducing diabetic hypercholesterolemia.

### Example 5

### Exposure to Nitraria retusa Significantly Increases Expression of Genes in the Liver which Regulate Energy Homeostasis (Glucose and Lipid Metabolism) in Diabetic Mice

Peroxisome Proliferator-Activated Receptor Alpha (PPAR alpha), acts as a master regulator of fatty acid oxidation. Sterol Regulatory Element Binding Protein-1c (SREBP-1c) is a transcription factor that controls genes involved in cholesterol uptake and biosynthesis. Fatty Acid Synthase (FAS) expression is strongly correlated with insulin sensitivity.

To assay the in vivo effects of Nitraria retusa extracts on genes which regulate energy homeostasis, Nitraria retusa extracts were administrated orally to mice and expression of said genes were determined by real-time PCR.

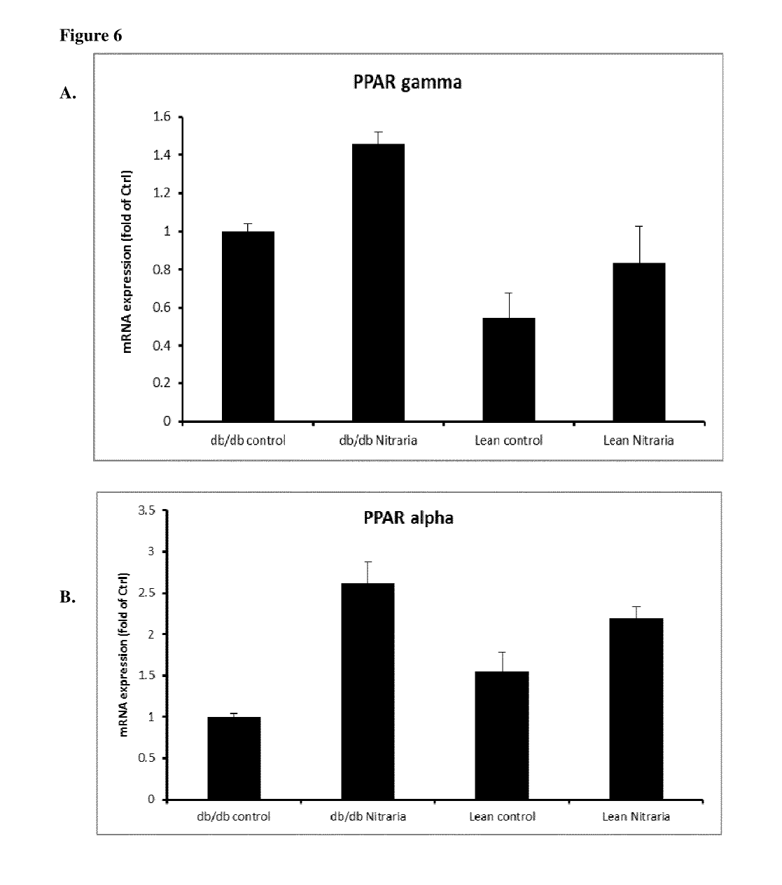

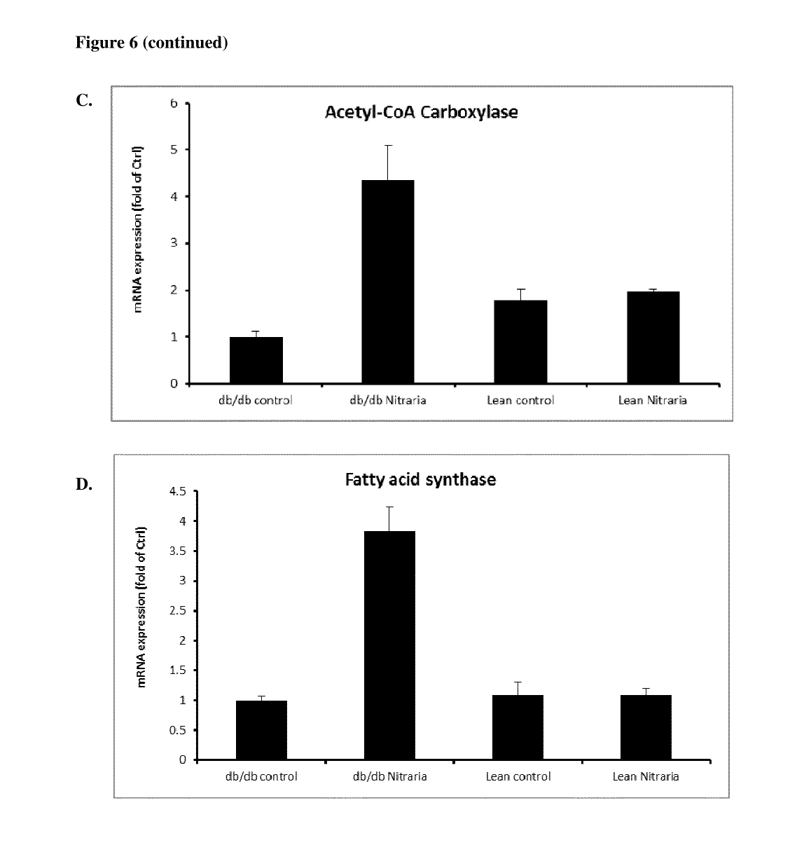

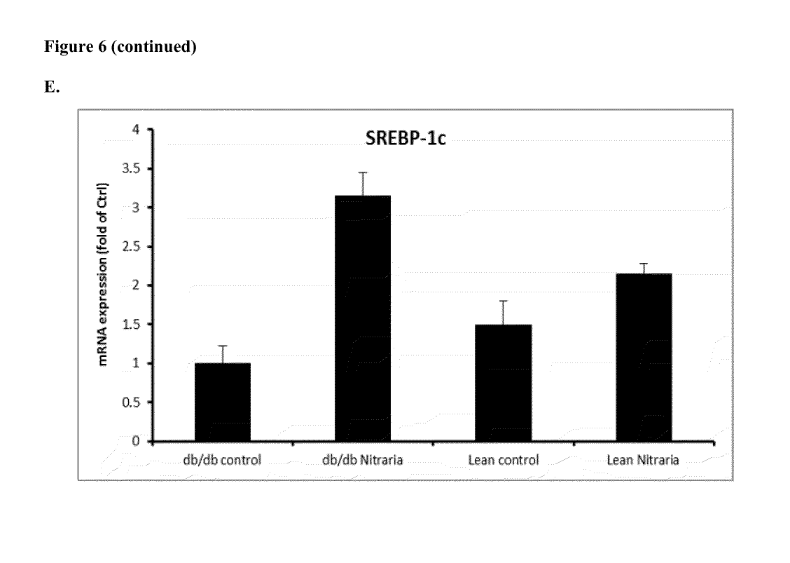

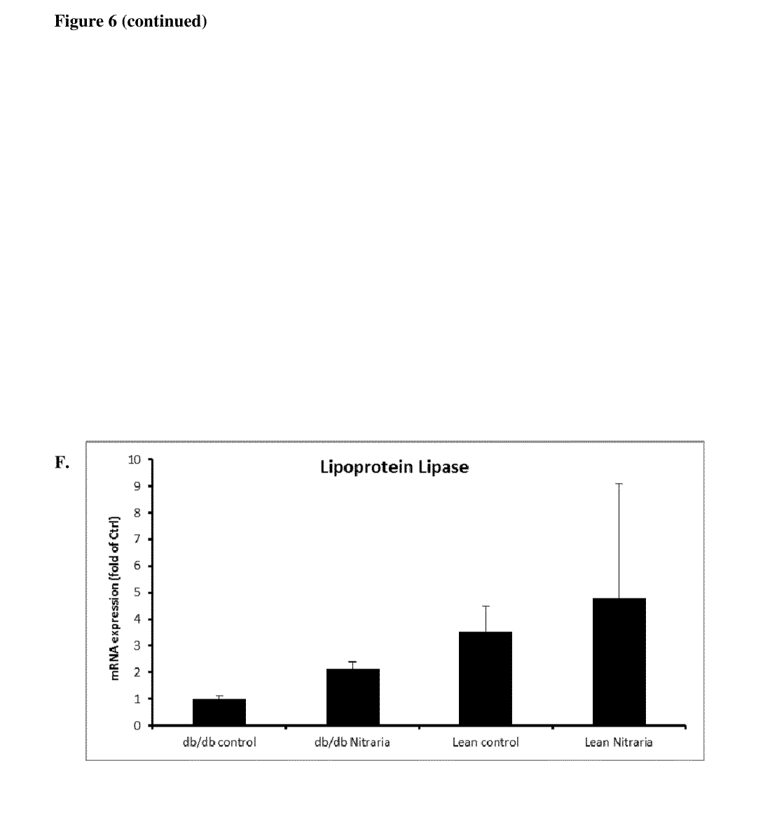

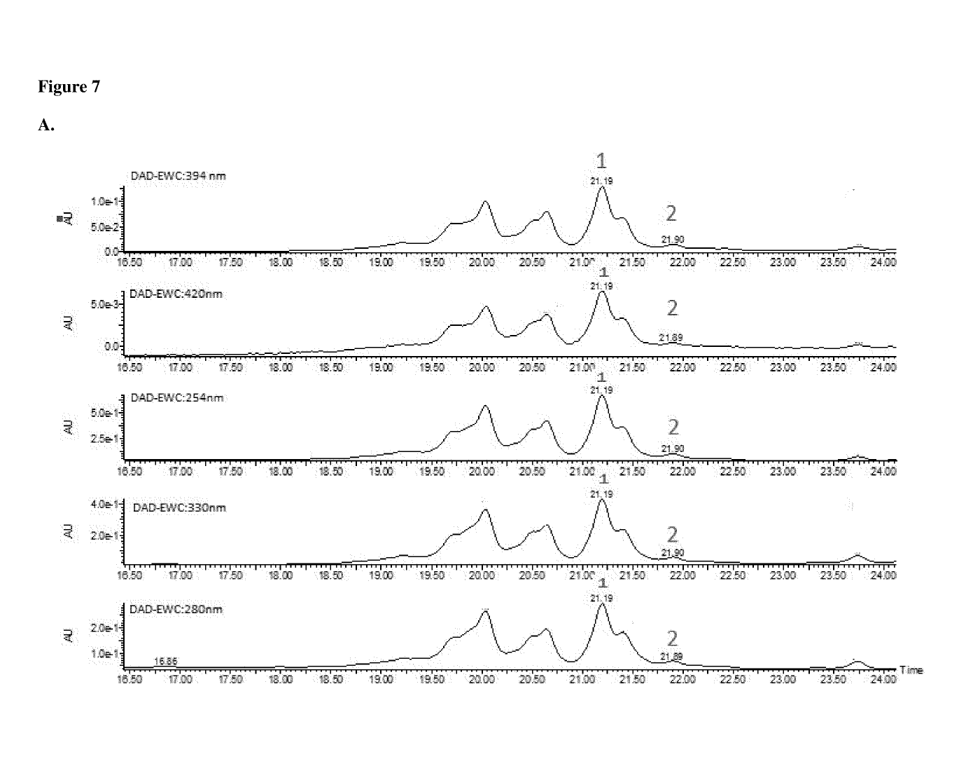

As shown FIGS. 6A-6F, oral administration of a Nitraria retusa extract significantly increased expression of PPAR gamma, PPAR alpha, Acetyl-CoA carboxylase (ACC), FAS, SREBP-1c, and Lipoprotein lipase in diabetic mice models.

These data indicate that in vivo administration of a Nitraria retusa extract is effective in enhancing of energy metabolism in the liver which treats or ameliorates obesity.

### Example 6

### Search for New Anti-Diabetic and Anti-Obesity Compounds in Nitraria retusa Extracts

The individual compounds present in Nitraria retusa extracts can be determined and isolated using procedures well-known in the art, e.g., HPLC, LC-MS, MS analyses.

Once an individual compound has been isolated from a Nitraria retusa extract, the compound can be tested using any of the protocols described herein to determine its effectiveness in inhibiting preadipocyte differentiation, reducing diabetic hypercholesterolemia, inhibiting lipid droplet accumulation in an adipocyte, and enhancement of energy metabolism in the liver and generally for treating or ameliorating obesity.

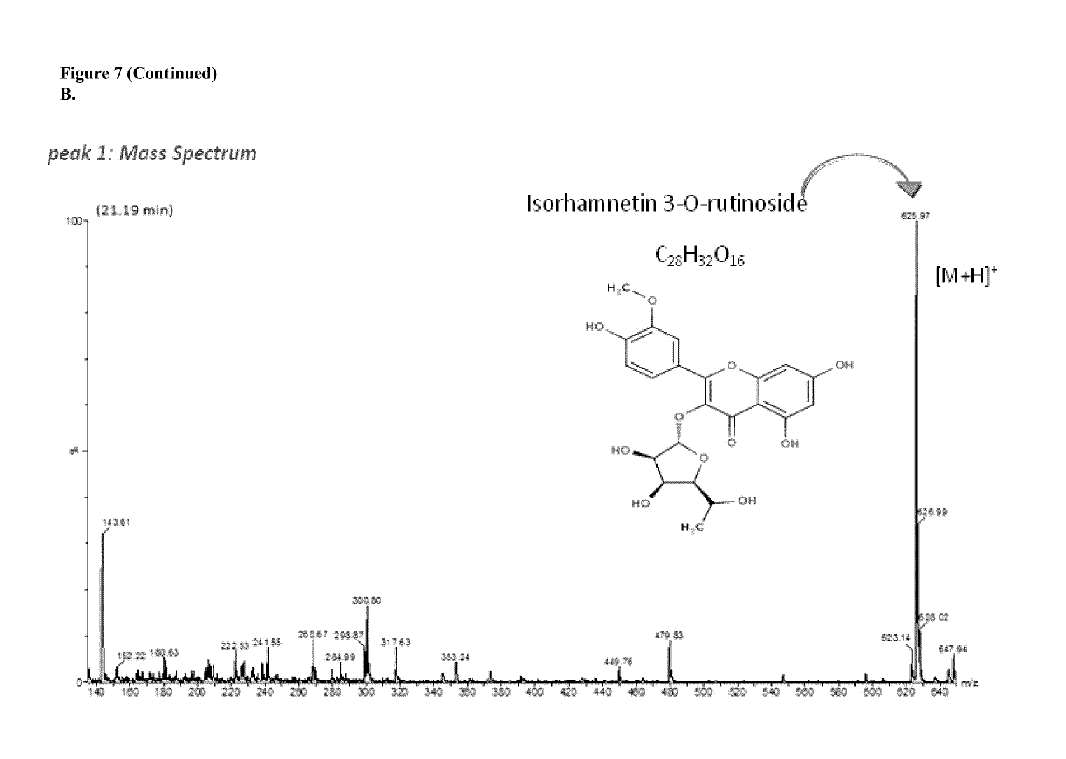

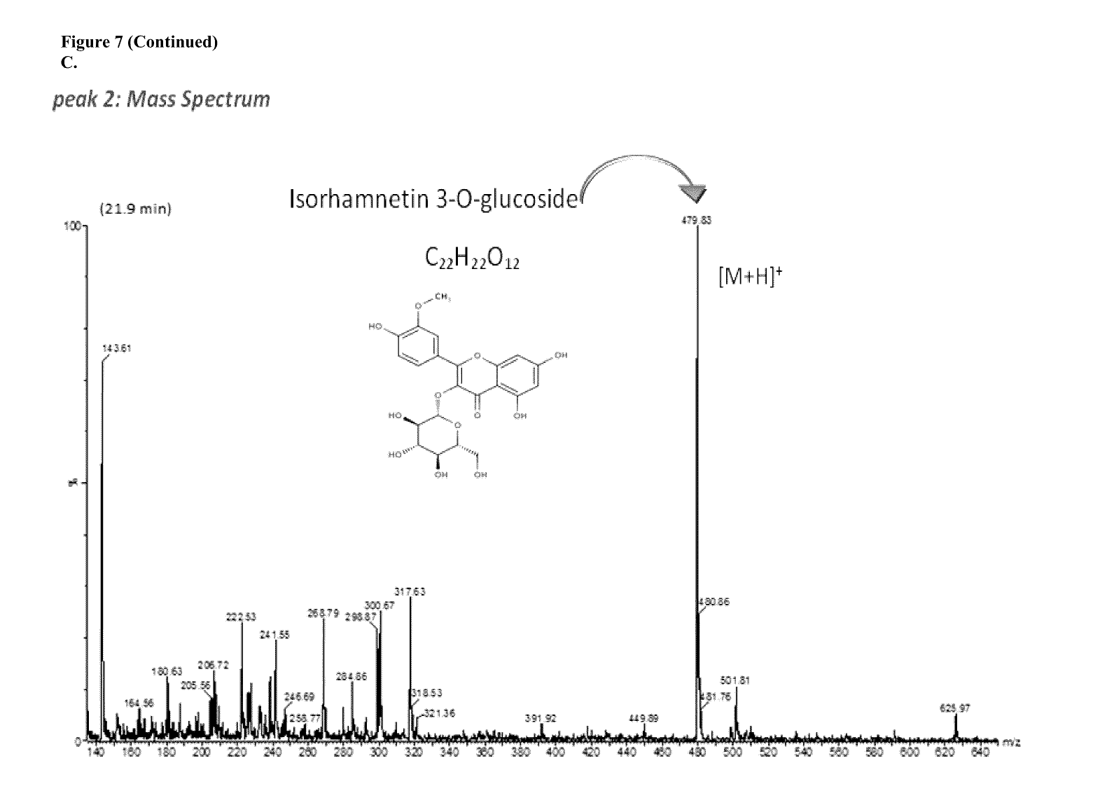

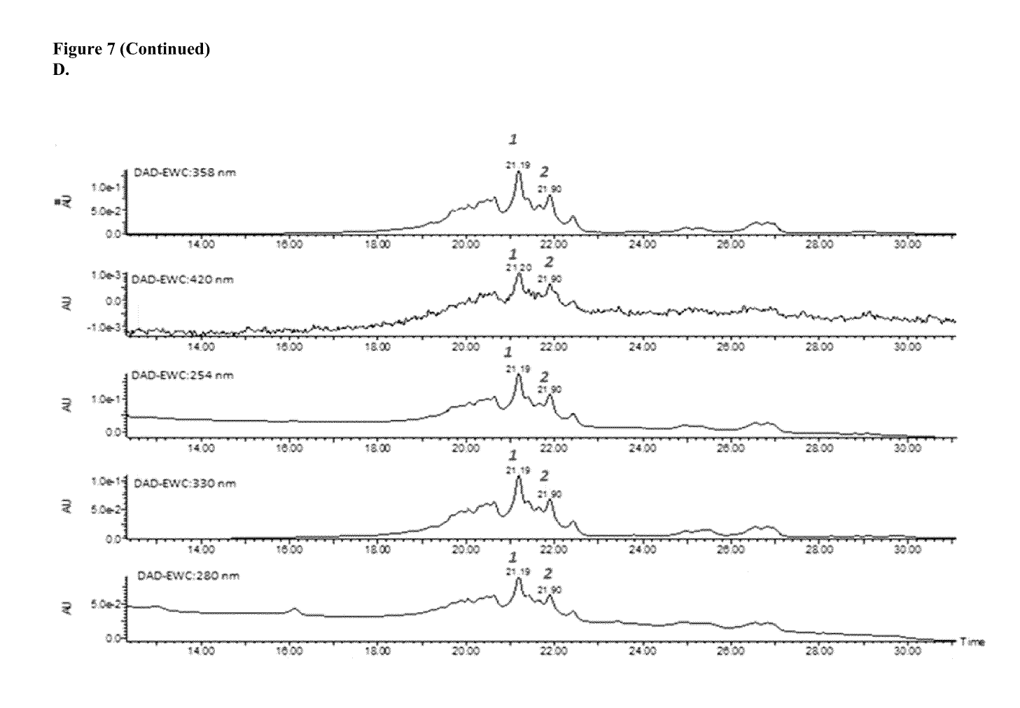

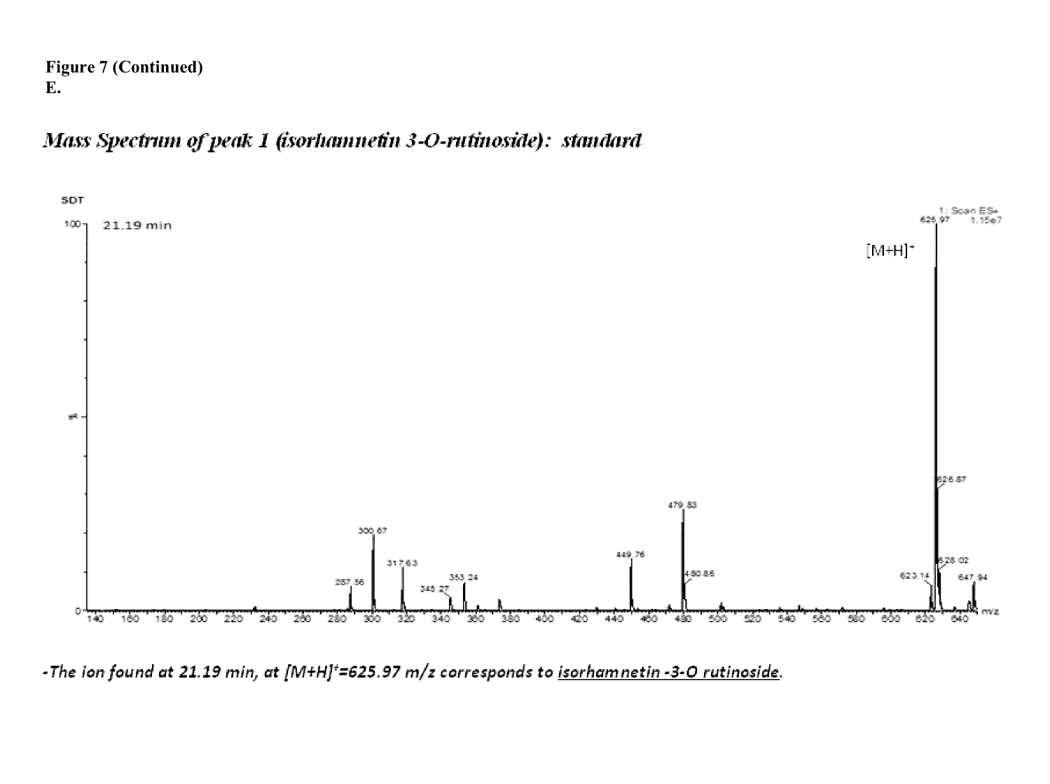

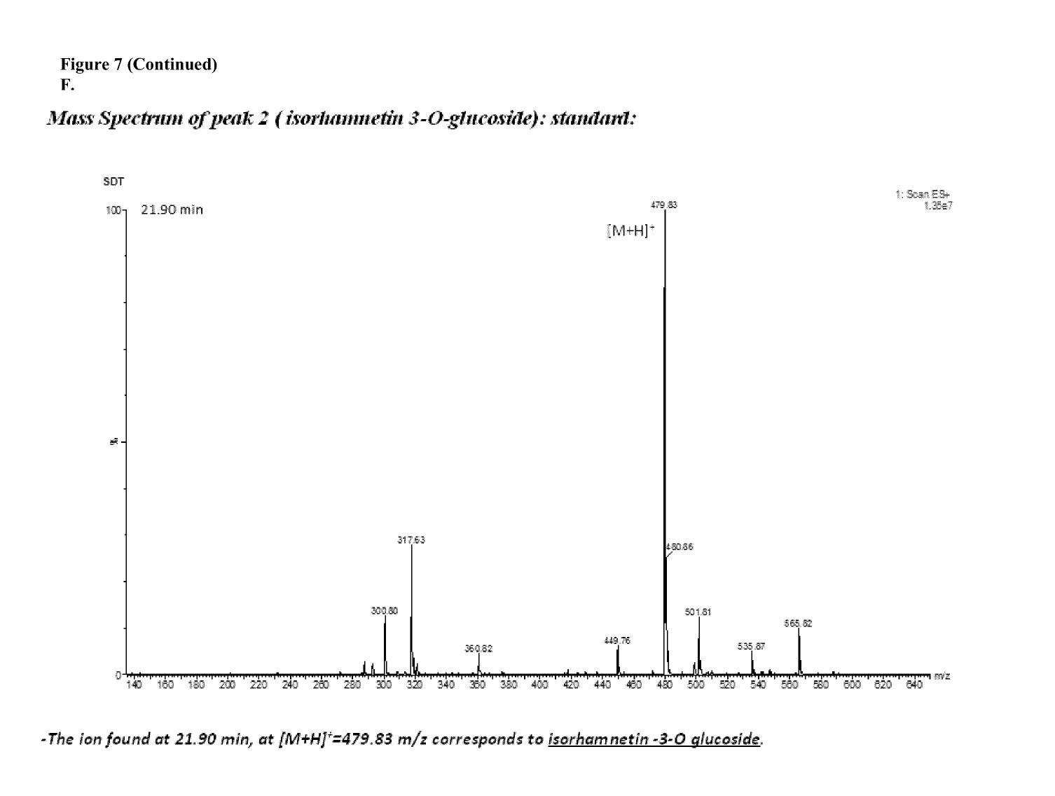

Indeed, as shown in FIG. 7, individual compounds can be identified and isolated from Nitraria retusa extracts using Liquid chromatography-mass spectrometry (LC-MS). FIGS. 7A-7C show the presence of isorhamnetin 3-O-rutinoside and isorhamnetin 3-O-glucoside which is confirmed by LC-MS using isorhamnetin 3-O-rutinoside and isorhamnetin 3-O-glucoside standards (FIGS. 7D-7F).

Obesity is one of the major health concerns in the Twenty-First Century and is one of the leading causes of preventable death. It is known to be a strong risk factor for Type 2 Diabetes. Due its complex etiology, treatment of obesity is difficult and challenging. Substantial progress has been made concerning our knowledge of bioactive components in plants and their link to obesity. Disclosed above is in vitro and in vivo data that Nitraria retusa extracts are useful for treating obesity and prevent metabolic syndrome-related diseases (e.g., hypercholesterolemia). These extracts can be included in food products, dietary supplements, and pharmaceutical compositions thereby providing bioactive compounds to a subject in need.

The adipocyte is the primary site of energy storage and it accumulates triglycerides during nutritional excess. Recent reports have outlined the mechanisms of proposed anti-obesity including decreased energy intake and increased energy expenditure, decreased pre-adipocyte differentiation and proliferation, decreased lipogenesis and increased lipolysis, and fat oxidation. Most excess energy is stored in the form of triglycerides in adipose tissue, and increased adipose tissue mass arises through an increase in the hypertrophy (cell size increasing) and hyperplasia (cell number increasing) which explains the interest in adipocyte proliferation and differentiation. Here, Nitraria retusa extracts are shown to modulate adipocyte hypertrophy rather than hyperplasia.

In contrast, Isorhamnetin, a well-known compound that inhibits adipogenesis by interfering with differentiation of adipose stem cells. Isorhamnetin represses adipogenesis in 3T3-L1 cells by inhibiting 3T3-L1 adipocyte differentiation by reducing C/EBP-α and PPAR-γmRNA levels. Isorhamnetin also reduces adiponectin expression and secretion, which may contribute to decreased adipocyte differentiation, thereby improving diet-induced obesity.

In recent decades, there has been increasing interest in Western Medicine for phytochemical combinations, which have been fundamental in traditional systems of herbal medicine. Combinations of some compounds present in a plant synergistically increase the herbal medicine's therapeutic activity. For example, in vitro and in vivo studies have shown that the polyphenol quercetin helps increase the polyphenol Resveratrol bioavailability resulting in synergistic anti-obesity activity. Although not bound by theory, the Nitraria retusa extracts disclosed herein likely have superior activity due to synergy brought about by the combination of compounds included in an extract.

In this regard, the Nitraria retusa extracts disclosed herein are likely rich in naturally present flavonoids and especially in Isorahmnetin and also Isorhamnetin-3-O-glucoside, Isorhamnetin-3-O-rutinoside, and Isorhamnetin-3-O-robinobioside, as shown in FIG. 7.

As described above, oral administration of a Nitraria retusa extract significantly reduces LDL cholesterol (the “bad cholesterol”) levels in diabetic mice models. Diabetes increases oxidative stress and levels of plasma total cholesterol and LDL cholesterol; these increased cholesterol levels are caused by diabetic dyslipidemia. The Nitraria retusa treated groups had significantly lower levels of plasma LDL cholesterol when compared to control groups. Thus, Nitraria retusa treatment reduces the risk of diabetic hypercholesterolemia.

As described above, oral administration of a Nitraria retusa extract did not significantly affect glucose and triglycerides levels. This is likely due to the duration of the disclosed experiments. It is known that energy is first stored by glucokinase activity in the liver to form glycogen whereas storage as triglyceride occurs later. Indeed, carbohydrates are stored as glycogen in the liver and muscles, but as these storage sites are filled, excess glucose is converted into triglycerides and stored in adipose tissue. Additionally, Nitraria retusa may regulate glucokinase activity in the liver.

Another method for treating obesity involves activating genes critical to lipid metabolism.

Peroxisome Proliferator-Activated Receptors (PPARs) are ligand-activated transcription factors and members of the nuclear hormone receptor superfamily, which regulate energy homeostasis (glucose and lipid metabolism), inflammation, proliferation and differentiation. In particular, PPAR alpha acts as a master regulator of fatty acid oxidation by controlling the transcription of its target gene. Consistent with this function, PPAR alpha is mainly expressed in tissues with high lipid catabolic capacities, such as the liver, muscle, and brown adipose tissue.

It has been reported that the activation of PPAR alpha enhances fatty acid oxidation in the liver and decreases the levels of circulating and cellular lipids in obese diabetic patients. In general, the accumulation of triglycerides in the liver is due to an imbalance between the availability of hepatic triglycerides for export and the exporting capacity of the liver via very low density lipoproteins (VLDLs).

In obesity and diabetes, the amounts of hepatic triglyceride available for export are increased because of enhanced uptake of unesterified fatty acids from plasma and or because of increased de novo fatty acids synthesis. Thus, the fatty acids are then incorporated into triglycerides. The combined process of fatty acid and triglyceride synthesis is called lipogenesis.

Fatty Acid Synthase (FAS) expression is strongly correlated with insulin sensitivity. The expression of FAS is higher in normal glucose tolerant subjects compared with subjects with impaired glucose tolerance.

Thiazolidinediones (TZDs) are agonists for Peroxisome Proliferator-Activated Receptor gamma (PPAR-γ), which promotes adipocyte differentiation. Treatment of patients with these drugs results in weight gain and an increase in fat mass accompanied by a shift of fat from the visceral to the subcutaneous depot, resulting in improved insulin sensitivity. In diabetes, inadequate glucose transport and metabolism as the consequence of insulin deficiency (Type 1) and insulin resistance (Type 2) results in fatty acid oxidation becoming almost the exclusive energy source for hearts.

Two enzymes that provide nonesterified fatty acid substrate for triglyceride synthesis are FAS and Lipoprotein Lipase (LPL); FAS regulates de novo lipogenesis from acetyl-CoA, malonyl CoA, and NADPH and is expressed at high levels in adipose tissue, liver, and lung. It has been reported, both FAS and LPL are increased by feeding; systemic overexpression of LPL in rabbits results in increases in whole body insulin sensitivity. LPL activity changes dramatically in various tissues in response to energy requirements. LPL overexpression prevents the development of diet-induced hypertriglyceridemia and hypercholesterolemia and decreases VLDL and LDL levels. Thus, in many studies cholesterol levels were decreased in LPL transgenic mice after cholesterol loading, so LPL plays an important role in the determining plasma cholesterol levels.

Sterol Regulatory Element Binding Protein-1c (SREBP-1c) is a transcription factor that controls genes involved in cholesterol uptake and biosynthesis. SREBP-1c has been shown to control expression of genes involved in the lipogenic pathway. SREBP-1c itself is rapidly induced by treatment of hepatocytes with insulin, providing a pathway for insulin mediation of enhanced lipogenic gene transcription. Consistent with these observations, mice with a deletion of the SREBP-1 gene have an impaired ability to fully respond to a high carbohydrate diet.

In vivo experiments using hamsters fed a cholesterol-rich diet also demonstrated decreased nuclear forms of SREBP-1 and -2 in liver. SREBP-1c is important in maintaining the basal level of transcription of Acetyl-CoA carboxylase (ACC) and FAS. SREBP-1c gene expression and protein activity are both directly subject to significant regulation by dietary and hormonal factors and it is likely an important mediator of insulin action in the liver.

Aberrant expression of SREBPs in mice results in metabolic syndromes with physiologic effects similar to specific disorders of lipid metabolism in humans. It has been demonstrated that SREBP-1c mRNA levels decreases in rats treated with streptozotocin (to induce diabetes) and insulin administration reverses this effect. Because the activation potential for SREBP-1-c is significantly lower than either SREBP-1a or SREBP-2, its over-expression in liver results in a much lower level of activation for genes of both fatty acid and cholesterol metabolism and a correspondingly lower level of accumulation of fatty acids and cholesterol.

Peroxisome proliferator-activated receptor-α (PPARα) plays a key role in the induction of the fatty acid oxidation enzymes in both peroxisomes and mitochondria, and the response elements have been identified in key enzymes, such as acyl-CoA oxidase and carnitine palmitoyltransferase-1. Free fatty acids directly bind and activate PPARα. Taken together, excess Highly Unsaturated Fatty Acids (HUFAs) are likely to induce their own oxidation machinery via a feed-forward mechanism, which is mediated by PPARα.

The results reported above were obtained using the following methods and materials.

### Plant Sampling

The halophytic species Nitraria retusa was selected in this study. Shoots collect was conducted in August 2010 from salt flat “Sabkha Elkelbia” located at N 35 48 44, E 10 09 06 of Kairouan. This locality is characterized by a semi-arid climate with less rainfall <200 mm/year and higher salinity mean (20 g/l). The collected samples were rinsed with distilled water, kept in laboratory temperature, oven dried at 60° C., and then ground finely using a ball mill type “Dangoumeau”. The plant powder obtained was stored at room temperature for different analyses.

### Extraction Methods

70% ethanol extraction and hot water extraction of the above-mentioned Nitraria retusa plant powder were conducted at 10% (w/v: weight of powder to volume of ethanol or water). The ethanol extract was prepared by combining the powder and ethanol and kept in the dark at room temperature for 2 weeks, with shaking at least once a day. The hot water extract was prepared by combining the powder and water and then autoclaving the mixture at 105° C. for 15 min. Liquid fractions were collected, filtered through 0.22 μm filter (MILLIPORE, U.S.A.), and concentrated using a speedvac (SCRUM Inc., Japan).

### Cell Culture

Murine 3T3-L1 preadipocytes (RIKEN Tsukuba, Japan) were purchased from the American Type Culture Collection (ATCC, Manassas, Va., USA) and cultured in 75-cm² tissue culture flasks in Dulbecco's modified Eagle's medium (DMEM) supplemented with 10% FBS and 1% penicillin (5000 μg/mL)-streptomycin (5000 IU/mL) solution in a humidified atmosphere at 37° C. and 5% CO₂. Cells were subcultured every 3 days. Cell passage was carried out at 80% confluence at one on two ratio using 0.25% trypsin (1 mM EDTA).

### In Vitro Assay: Induction of Adipogenesis in 3T3-L1 Cells

3T3-L1 cells were cultured until confluent. Two days after reaching confluence (Day 0), the culture medium was changed to a differentiation-induction medium containing 1/10 insulin solution, 1/10 dexamethasone solution, and 1/10 3-Isobutyl-1-methylxanthine solution in DMEM with 10% fetal bovine serum for 2 days. Then, cells were maintained in a differentiation-maintenance medium containing only 1/10 insulin solution.

### In Vitro Assay: Pre-Adipocytes Differentiation Procedure

3T3-L1 pre-adipocytes in 80% confluent cell layers were seeded in 96-well plates at 1×10⁵ cells/ml and cultured for an additional two days. Two days after reaching confluence (Day 0), cells were incubated with a differentiation cocktail (MDI) containing 1/10 insulin solution, 1/10 dexamethasone solution, and 1/10 33-Isobutyl-1-methylxanthine solution in standard culture medium DMEM (with 10% fetal bovine serum FBS and 1% Penicillin/Streptomycin (PS)), followed by additional 48 hours with standard culture medium containing insulin alone. The differentiation-maintenance medium was changed every two days until the cells were harvested. To test the effects of Nitraria retusa ethanol and hot water extracts using concentration range 25, 50, 100, 200, or 400 μg/ml on adipogenesis, samples were added to the differentiation-induction and differentiation-maintenance media until the cells were harvested.

In vitro assay: Oil-Red-O Staining Procedure

The staining procedure was conducted according to the Adipogensis Assay Kit (Cayman Chemical Company). First, media from the wells was removed. 75 μl of lipid Droplets Assay Fixative (10×) was added to each well and incubated for fifteen minutes. Wells were twice washed with 100 μl of wash solution for five minutes each. After complete drying of wells, 75 μl of Oil Red O working solution was added to all wells including the background wells containing no cells and incubated for 20 minutes. Next, all Oil Red O solution was removed and cells were washed with distilled water several times until the water contained no visible pink color. The wells were twice washed with 100 μl of wash solution for five minutes each. Microscope images were taken to visualize pink to red oil droplets staining in differentiated cells. When the wells were completely dry, 100 μl of dye extraction solution was added to each well. Finally, and after gently mixing for 15-30 minutes, the absorbance was read at 490 nm with a 96-well plate reader.

### Animals and Experimental Design

Five-week-old, male BKS.Cg-Dock7m+/+Lepr^{db}/J mice were purchased from Jackson Laboratory (USA) and maintained under a light cycle (12 hour light/dark), fed with mouse chow (purchased from PMI Nutrition International) which contained 3.13 Kcal/g with 10.5% calories from fat, 25% from protein, and 64.5% from carbohydrates. The animals were allowed one week to acclimatize themselves to the housing conditions before the beginning of an experiment. Mice were randomly divided in 4 groups, 2 groups of db/db mice, one of which was orally administrated a daily dose of Nitraria retusa extract (at 50 mg/kg of body weight) and 2 groups of lean mice (db/+ littermates), one of which was orally administrated a daily dose of Nitraria retusa extract (at 50 mg/kg of body weight). Body weight and food intake were measured daily and weekly at regular times. After four weeks of administration, mice were killed and blood was taken for biochemical analysis. All procedures were performed in accordance with the Ethics Animal Care and Use Committee of the University of Tsukuba, Japan.

### Measurements of Body, Organ, and Fat Weight

Body weight was measured once a day during the feeding period. Internal organs were dissected and weighed. Fat tissue samples and liver samples also were stored until they were analyzed for further experiments.

### Measurements of Glucose, Triglyceride and Cholesterol Levels

After 4 weeks of treatment, blood samples were collected and centrifuged at 3,000 rpm for fifteen minutes at 4° C., and serum glucose, triglyceride (TG), total cholesterol (TCHO), high-density lipoprotein (HDL) and low-density lipoprotein (LDL) levels were measured according to the manufacturer's instruction.

### Total RNA Extraction:

Liver samples (50 mg) were prepared and homogenized with a Polytron homogenizer. Then total RNA was purified using the ISOGEN kit (Nippon GeneCo. Ltd., Japan) following the manufacturer's instructions.

cDNA Synthesis:

Total RNA was quantified using Thermo scientific Nanodrop 2000 (USA), and the reverse transcription reactions were performed using the Superscript III reverse transcriptase kit (Invitrogen, Carlsbad, Calif., USA) using 1 μg of total RNA. Briefly, RNA was denatured by incubation at 65° C. for five minutes, with 1 μL oligo (dT) primers, and chilled at 4° C. Then SuperScript III reverse transcriptase was added and the reaction mix was then incubated at 42° C. for sixty minutes, then ten minutes at 70° C.

### Real-Time PCR:

The expression of PPAR gamma, PPAR alpha, LPL, FAS, ACC, and SREBP-1-c were determined by real-time PCR using β-actin (a housekeeping gene) as a reference control. Primers and TaqMan probes used for these experiments were purchased from Applied Biosystems. Primers were inventoried gene expression assays. TaqMan real-time PCR amplification reactions were performed in a 20 μl reaction mixtures containing: 10 μl of TaqMan Universal PCR Master Mix UNG (2×), 9 μl of template cDNA (100 ng μl-1) and 1 μl of the corresponding primer/probe mix, using an AB 7500 fast real-time system (Applied Biosystems). For the amplification, the following cycling conditions were applied: 2 min at 50° C., 10 min at 95° C., and 40 cycles of 15 s at 95° C./1 min at 60° C.

### Liquid Chromatography-Mass Spectrometry (LC-MS)

HPLC and LC-MS were performed according to manufacture's instructions.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A composition formulated for oral administration comprising a therapeutically effective amount of a Nitraria retusa extract or a purified fraction thereof, wherein the composition is a food product, a dietary supplement, or a pharmaceutical composition.

2. The composition of claim 1, wherein the extract is an ethanol extract.

3. The composition of claim 1 formulated as a tablet, a capsule, a softgel, a liquid gel, a pill, a granule, a syrup, a paste, a powder, a lozenge, a concentrate, a liquid, or a dry syrup.

4. The composition of claim 1, comprising a therapeutically effective amount of a Nitraria retusa extract.

5. The composition of claim 1, comprising a therapeutically effective amount of a purified fraction of a Nitraria retusa extract.

6. The composition of claim 5, wherein the purified fraction is an isolated compound.

7. A method for inhibiting preadipocyte differentiation, the method comprising administering to a preadipocyte an effective amount of a Nitraria retusa extract or a purified fraction thereof.

8. The method of claim 7, wherein the extract is an ethanol extract.

9. The method of claim 7, wherein the preadipocyte is in vitro.

10. The method of claim 7, wherein the preadipocyte is in a subject being a human, a pet, or a livestock.

11. The method of claim 10, wherein the human is a diabetic or a prediabetic.

12. The method of claim 10, wherein the Nitraria retusa extract or a purified fraction thereof is in a composition being a food product, a dietary supplement, or a pharmaceutical composition and being formulated for oral administration.

13. The method of claim 12, wherein the composition is a food product selected from the group consisting of a beverage, a bread, a candy, a cereal, a chocolate, a coffee, a condiment, a cookie, a cracker, an energy drink, a gel, an ice cream, a jelly, a juice, a milk-containing beverage, a nutritional bar, a pasta, a paste, a processed fruit, a processed grain, a processed meat, a processed vegetable, a pudding, a snack bar, a soft drink, a tea, a yogurt, an animal feed, and a pet food.

14. The method of claim 13, wherein the food product is a tea.

15. The method of claim 12, wherein the composition is a dietary supplement or a pharmaceutical composition and is formulated into one or more forms selected from the group consisting of: a tablet, a capsule, a softgel, a liquid gel, a pill, a granule, a syrup, a paste, a powder, a lozenge, a concentrate, a liquid, and a dry syrup.

16. The method of claim 11, wherein inhibiting fat accumulation in the preadipocyte treats, ameliorates obesity or increases fat burning.

17. A method for reducing diabetic hypercholesterolemia, the method comprising administering to a subject in need thereof an effective amount of a composition comprising a Nitraria retusa extract or a purified fraction thereof.

18. The method of claim 17, wherein the subject is a human, a pet, or a livestock.

19. The method of claim 18, wherein the human is a diabetic or a prediabetic.

20. The method of claim 18, wherein the composition is a food product, a dietary supplement, or a pharmaceutical composition and being formulated for oral administration.

21. The method of claim 20, wherein the food product selected from the group consisting of a beverage, a bread, a candy, a cereal, a chocolate, a coffee, a condiment, a cookie, a cracker, an energy drink, a gel, an ice cream, a jelly, a juice, a milk-containing beverage, a nutritional bar, a pasta, a paste, a processed fruit, a processed grain, a processed meat, a processed vegetable, a pudding, a snack bar, a soft drink, a tea, a yogurt, an animal feed, and a pet food

22. The method of claim 20, wherein the food product is a tea.

23. A method for inhibiting lipid droplet accumulation in an adipocyte, the method comprising administering to an adipocyte an effective amount of a Nitraria retusa extract or a purified fraction thereof.

24. A method for enhancement of energy metabolism in the liver, the method comprising administering to a subject in need thereof an effective amount of a composition comprising Nitraria retusa extract or a purified fraction thereof.

25. The method of claim 24, wherein enhancement of energy metabolism in the liver is enhancement of gene expression related to lipid metabolism.

26. The method of claim 25, wherein gene expression of Fatty Acid Synthase (FAS), peroxisome proliferator-activated receptor alpha (PPAR-α), peroxisome proliferator-activated receptor gamma (PPAR-γ), or sterol regulatory element binding protein-1c (SREBP-1c) is enhanced.

27. The method of claim 25, wherein enhancement of energy metabolism in the liver treats or ameliorates obesity or increases fat burning.
